(19) United States
(12) Patent Application Publication
(10) Pub. No.: US 2020/0108015 A1
(43) Pub. Date: Apr. 9, 2020

(54) Sustained Release Nitric Oxide in Intestinal Tract

(71) Applicant: SKLAR PHARMA LLC, Livingston, NJ (US)

(72) Inventor: Michael Farber, Livingston, NJ (US)

(21) Appl. No.: 16/150,536

(22) Filed: Oct. 3, 2018

(51) Int. Cl. A61K 9/16; A61K 47/32; A61K 47/18; A61P 9/10; A61P 9/12

(52) U.S. Cl. CPC A61K 9/1682; A61K 47/32; A61K 9/1635; A61P 9/12; A61K 9/1641; A61K 47/183; A61P 9/10; A61K 9/1617

(57) ABSTRACT

Nitric oxide levels in the blood stream are maintained while delivering, in sustained release, medication or therapeutic agent such as an NSAID (e.g. aspirin). This is accomplished by processing, at room temperature or in a low pressure environment without degradation, an amino acid ester with a nitric acid releasing group in an ethyl cellulose-based or other sustained release polymer delivery system. In this manner, in the blood stream nitric oxide levels are maintained at a level to only produce normotension because the nitric oxide is released only during hypertensive moments. The level of nitric oxide release metabolites reaches significance and physiological relevance anywhere between 12 and 48 hours depending upon the individual being dosed level of activity, ambient systolic blood pressure and numerous other factors that create the hypertensive environment that releases nitric oxide from the nitric oxide releasing amino acid ester.

### FIELD OF THE DISCLOSED TECHNOLOGY

The subject matter disclosed generally relates to amino acid ester compounds comprising at least one nitric oxide releasing group and a method of slow release thereof.

### BACKGROUND OF THE DISCLOSED TECHNOLOGY

Nitrates, when ingested by humans, are converted orally into nitrite by salivary secretions, then into nitric oxide in the gastrointestinal tract. This protects the tract from gastric toxicity of non-steroidal anti-inflammatory drugs (NSAIDs) and other drugs or agents. For example, aspirin is associated with upper gastrointestinal (GI) side effects including ulcers and bleeding. Large doses of aspirin, if taken once a day, interfere with platelet thromboxane production while in the blood stream (though the half life of aspirin is only about 30 minutes. Aspirin also interferes with the secretion of prostacyclins and other prostanoids which is counterproductive causing side effects of ulceration in the gastro intestinal tract. Some NSAIDs are even more ulcerative than aspirin, and additionally, increase blood pressure. Therefore during consumption of NSAIDS it is advantageous to have a continuous supply of nitrates into saliva from a constant release of nitric oxide in the blood which would result in both lowering blood pressure and increase salivary nitrate supply. This salivary nitrate supply is converted to nitrite in saliva and thereafter to nitruc oxude in the gastric environment providing gsstric protection. Nitric oxide also plays fundamental roles in other physiological processes. Maintaining balance within a human subject promotes or restores both cardiovascular health and healthy functioning of other physiological organ systems.

In U.S. Pat. No. 9,085,508 to the same inventor, which is hereby incorporated by reference in full, nitric oxide releasing compounds were disclosed. However, it was now unexpectedly found that as the dosage of a delivered amino acid releasing nitric oxide ester increased, nitric oxide levels in the blood failed to increase as much as desired. In fact, there seemed to exist no relationship between dose and response after a certain single dosing level was achieved of approximately 0.25 mg/kg. That due to the nature of the nitric oxide releasing amino acid esters, after a certain amount of nitric oxude release vasodilation occurs that causes a down regulation of the enzyme metabolizing the nitric oxide donating moiety. This dose response only occurred during hypertensive episodes, such as after consuming a meal. The dose response is due to an equilibrium state that is reached the delivered molecule is metabolized by glutathione transferase, an enzyme activated by sheer stress during hypertensive episode, releasing said nitric oxide. As the nitric oxide is released, vasodilation starts to occur reducing sheer stress and, in turn, deactivating the metabolic enzyme system, glutathione transferase. As vasotension returns to the normotensive state,—the metabolism of the delivered molecule falls to around 3% of a basal metabolic rate of the endothelial gst system delivering only a negligble effect during normotensive condition. This means that in a normotensive state, only limited amounts of nitric oxide are released from a single dose of the molecule, no matter the dosage size and exhibit minimal physiological effect. This amounts to less than a 10% formation of nitrates and nitrites, rather than what would be expected through typical nitrate donor metabolism or other nitric oxide donors. As can be seen in FIG. 3 of the '508 patent, vasodilation reaches a maximum threshold during an “induced” hypertensive event, namely physical exertion. Therefore, unless dosing in a normotensive person occurs within a given time frame (before a hypertensive episode) no release of nitric oxide foccurs. During a single isolated transient hypertensive episode in anotherwise normotensive individual, an amount of nitric oxide less than physiologically relevant would still only be released.

What is needed is a way to increase and sustain the release of nitric oxide and hence circulating nitrate and nitrite levels, and thereby gastro-protective nitric oxide levels in the gastrointestinal tract throughout the delivery period or period of effect of another medication such as an NSAID.

### SUMMARY OF THE DISCLOSED TECHNOLOGY

To be able to provide both normotensive and hypertensive individuals with a physiologically effective dose of nitric oxide from the inventive compounds in combination with other therapeutic agents only once or twice a day, a sustained release formulation that is activated several times daily in a normotensive subject must be administered. —The effects of eating, normal activity, and daily emotional stressors intermittently activate the metabolism of the circulating compound and produce more nitric oxide and hence add to the already accumulating nitrate pool. It should be noted that the compounds' release of nitric oxide decreases over several days as a new normotensive state is achieved through a new nitrate/nitrite balance that, based on testing to date, is expected to achieve a 4 to 6 mm Hg (millimeters of mercury) decrease in resting systolic blood pressure at its equilibrium point, much like the consumption of high nitrate vegetables can achieve in certain diets.

With respect to hypertensive individuals, a more constant release of nitric oxide takes place, but again, the metabolism of the molecule would slow down appreciably as the subject physiology approached a normotensive state so that in cases of both normotensive and hypertensive individuals, no hypotension occurs. The sustained release of nitric oxide is controlled both by the vasotensive environment of the individual and the release rate of the compound into the subject's circulatory system. Within 24 to 48 hours of dosing, the sustained release formulation produces—an increase of circulating nitrates such that an equilibrium state is achieved that is approximately 50 uM to 150 uM above the pre-dosing level for the subject. This equilibrium state also comes with a respective increase in circulating nitrite of between 600 to 1100 nM above predosing levels which would be physiologically relevant.

It should be understood that the release of nitric oxide in the endothelium creates other physiological effects. Numerous scientific research publications concerning nitric oxide and its signaling pathways have demonstrated physiological effects, either directly through nitrosylation, through augmenting cGMP levels, or through other intermediates and metabolites.

In other combinations, the sustained release of nitric oxide in the blood with other therapeutics is envisioned to have a positive and or protective effect upon renal function, cardiac function, endothelial function, or a positive effect upon red blood cell production, and fetal hemoglobin production with other therapeutics. The disclosed technology therefore applies to the sustained release of nitric oxide in combination with therapeutic (s) agent or agents that would benefit from the sustained release of nitric oxide either synergistically or antagonistically and the resultant increases in circulating nitrate and nitrites in the mammalian treated subjects.

In embodiments of the disclosed technology, a nitric oxide releasing compound, having an amino acid ester with at least one nitric oxide releasing group is used together with a polymer causing sustained release, as well as a controlled release carrier or enteric coating. The sustained release formulation of nitric oxide releasing amino acid esters can be formulated in 24 hour release micro beads and dosed concurrently with 24 hour sustained release aspirin microbeads to enhance anti thrombotic effects while diminishing or eliminating gastric ulceration. Other drugs delivered with the nitric oxide releasing amino acid esters are also within the scope of the disclosed technology.

The nitric oxide releasing amino acid ester, in embodiments of the disclosed technology, is released into the gut from 4 to 24 hours, entering the blood stream as a nitric oxide donating moiety, that releases nitric oxide triggered by a hypertensive state or event. This nitric oxide releasing amino acid ester is delivered in an amount between 50 to 250 milligrams per day in a sustained format in some embodiments. The polymer can be hydroxyethylcellulose and/or ethylcellulose having a combined weight of at least twice that of the drug. A microencapsulated release formulation of the compound can be made having a coating which is a majority ethylcelluose and a hydrophilic polymer in a ratio of about 20:1 and wherein a total weight of the coating is about 20% to 30% of the formulation. The mixture (any of the above) can be dissolved in a least polar alcohol, and dried at 35 degrees Celsius or less. The pressure can be atmospheric pressure or less.

The drug can be aspirin with a sustained release over a 24 hour period of time. The nitric oxide releasing amino acid ester is released over 24 hours into the gastro intestinal tract, then enters the blood stream where it releases nitric oxide, in a quantity calibrated to offset substantially all or all gastric toxicity caused by the aspirin during it's release. The amount of aspirin can be, in some embodiments, 150 to 375 milligrams or more.

The nitric oxide releasing amino acid ester is between 30% and 100% by weight, compared to the NSAID or aspirin and each NSAID or aspirin has a sustained release or normal release profile over a period of between 4 and 24 hours. As such, in embodiments of the disclosed technology, nitric oxide levels are substantially sustained during this release profile of the NSAID or aspirin. A polymer causing sustained release is selected from a group consisting of a hydroxypropyl methylcellulose ether, hydroxyethylcellulose, and ethylcellulose in some embodiments or can be selected from lipid polymers in other embodiments.

The following terms are defined below.

“Amino acid ester compound” is intended to mean the condensation product of an amino acid with mononitrated alkane ou alkene diol. As will be evident to those familiar to the art, the condensation reaction could also involve, but not limited to, dipeptides or tripeptides, nitrated alcohols containing aliphatic, alkyl or aromatic moieties, as well as other nitric oxide groups attached to the alkane or alkene diols. Amino acid or dipeptide reactions are favoured as well as the condensation reaction with short chain mononitrated alkane diols such as 1,3 propanediol or 1,4 butanediol.

“Cardiovascular disease or disorder” is intended to mean any cardiovascular disease or disorder known in the art, including, but not limited to, congestive heart failure, restenosis, hypertension (e.g. pulmonary hypertension, labile hypertension, idiopathic hypertension, low-renin hypertension, salt-sensitive hypertension, low-renin, salt-sensitive hypertension, thromboembolic pulmonary hypertension; pregnancy-induced hypertension; renovascular hypertension; hypertension-dependent end-stage renal disease, hypertension associated with cardiovascular surgical procedures, hypertension with left ventricular hypertrophy, and the like), diastolic dysfunction, coronary artery disease, myocardial infarctions, cerebral infarctions, atherosclerosis, atherogenesis, cerebrovascular disease, angina, (including chronic, stable, unstable and variant (Prinzmetal) angina pectoris), aneurysm, ischemic heart disease, cerebral ischemia, myocardial ischemia, thrombosis, platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neointimal hyperplasia following percutaneous transluminal coronary angiograph, vascular grafting, coronary artery bypass surgery, thromboembolic events, post-angioplasty restenosis, coronary plaque inflammation, hypercholesterolemia, embolism, stroke, shock, arrhythmia, atrial fibrillation or atrial flutter, thrombotic occlusion and reclusion cerebrovascular incidents, and the like.

“Thromboembolic events” include, but are not limited to, ischemic stroke, transient ischemic stroke, myocardial infarction, angina pectoris, thrombosis (for example, restenosis, arterial thrombosis, coronary thrombosis, heart valve thrombosis, coronary stenosis, stent thrombosis, graft thrombosis, and first and subsequent thrombotic stroke, and the like), thromboembolism (for example, pulmonary thromboembolism, cerebral thromboembolism, and the like), thrombophlebitis, thrombocytopenia, bleeding disorders, thrombotic occlusion and reocclusion and acute vascular events. Patients who are at risk of developing thromboembolic events, may include those with a familial history of, or genetically predisposed to, thromboembolic disorders, who have had ischemic stroke, transient ischemic stroke, myocardial infarction, and those with unstable angina pectoris or chronic stable angina pectoris and patients with altered prostacyclin/thromboxane A.sub.2 homeostasis or higher than normal thromboxane A.sub.2 levels leading to increase risk for thromboembolism, including patients with diabetes and rheumatoid arthritis.

“Diseases resulting from oxidative stress” is intended to mean any disease that involves the generation of free radicals or radical compounds, such as, for example, atherogenesis, atheromatosis, arteriosclerosis, atherosclerosis, vascular hypertrophy associated with hypertension, hyperlipoproteinaemia, normal vascular degeneration through aging, parathyroidal reactive hyperplasia, renal disease (e.g., acute or chronic), neoplastic diseases, inflammatory diseases, neurological and acute bronchopulmonary disease, tumorigenesis, ischemia-reperfusion syndrome, arthritis, sepsis, cognitive dysfunction, endotoxic shock, endotoxin-induced organ failure, and the like.

“Renovascular diseases” is intended to mean any disease or dysfunction of the renal system including, but not limited to, renal failure (e.g. acute or chronic), renal insufficiency, nephrotic edema, acute glomerulonephritis, oliguric renal failure, renal deterioration associated with severe hypertension, unilateral perechymal renal disease, polycystic kidney disease, chronic pyelonephritis, renal diseases associated with renal insufficiency, complications associated with dialysis or renal transplantation, renovascular hypertension, nephropathy, glomerulonephritis, scleroderma, glomerular sclerosis, and the like “Endothelial dysfunction” refers to the impaired ability in any physiological processes carried out by the endothelium, in particular, production of nitric oxide regardless of cause. It may be evaluated by, such as, for example, invasive techniques, such as, for example, coronary artery activity to acetylcholine or methacholine, and the like, or by noninvasive techniques, such as, for example, blood flow measurements, brachial artery flow dilation using cuff occlusion of the arm above or below the elbow, brachial artery ultrasonography, imaging techniques, measurement of circulating biomarkers, such as, asymmetric dimethylarginine (ADMA), and the like. For the latter measurement the endothelial-dependent flow-mediated dilation will be lower in patients diagnosed with an endothelial dysfunction.

“Methods for treating endothelial dysfunction” include, but are not limited to, treatment prior to the onset/diagnosis of a disease that is caused by or could result from endothelial dysfunction, such as, for example, atherosclerosis, hypertension, diabetes, congestive heart failure, and the like.

“Methods for treating diseases caused by endothelial dysfunction” include, but are not limited to, the treatment of any disease resulting from the dysfunction of the endothelium, such as, for example, arteriosclerosis, congestive heart failure, hypertension, cardiovascular diseases, cerebrovascular diseases, renovascular diseases, mesenteric vascular diseases, pulmonary vascular diseases, ocular vascular diseases, peripheral vascular diseases, peripheral ischemic diseases, and the like.

“Therapeutic agent” is intended to mean any therapeutic agent that can be used to treat or prevent the diseases described herein. “Therapeutic agents” include, for example, aldosterone antagonists, alpha-adrenergic receptor antagonists, angiotensin II antagonists, angiotensin-converting enzyme (ACE) inhibitors, antidiabetic compounds, anti-hyperlipidemic compounds, antioxidants, antithrombotic and vasodilator compounds, (.beta.-adrenergic antagonists, calcium channel blockers, digitalis, diuretics, endothelin antagonists, hydralazine compounds, H2 receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, and the like. Therapeutic agent includes the pharmaceutically acceptable salts thereof, pro-drugs, and pharmaceutical derivatives thereof including, but not limited to, the corresponding nitrosated and/or nitrosylated and/or alphatic or heterocyclic nitric oxide donor derivatives. Although nitric oxide donors have therapeutic activity, the term “therapeutic agent” does not include the nitric oxide donors described herein, since nitric oxide donors are separately defined.

“Prodrug” is intended to mean a compound that is made more active in vivo.

“Antioxidant” is intended to mean any compound that can react and quench a free radical.

“Angiotensin converting enzyme (ACE) inhibitor” is intended to mean compounds that inhibit an enzyme which catalyzes the conversion of angiotensin I to angiotensin II. ACE inhibitors include, but are not limited to, amino acids and derivatives thereof, peptides, including di- and tripeptides, and antibodies to ACE which intervene in the renin-angiotensin system by inhibiting the activity of ACE thereby reducing or eliminating the formation of the pressor substance angiotensin II.

“Angiotensin II antagonists” refers to compounds which interfere with the function, synthesis or catabolism of angiotensin II. Angiotensin II antagonists include peptide compounds and non-peptide compounds, including, but not limited to, angiotensin II antagonists, angiotensin II receptor antagonists, agents that activate the catabolism of angiotensin I, and agents that prevent the synthesis of angiotensin I from angiotensin II. The renin-angiotensin system is involved in the regulation of hemodynamics and water and electrolyte balance. Factors that lower blood volume, renal perfusion pressure, or the concentration of sodium in plasma tend to activate the system, while factors that increase these parameters tend to suppress its function.

“Anti-hyperlipidemic compounds” is intended to mean any compound or agent that has the effect of beneficially modifying serum cholesterol levels such as, for example, lowering serum low density lipoprotein (LDL) cholesterol levels, or inhibiting oxidation of LDL cholesterol, whereas high density lipoprotein (HDL) serum cholesterol levels may be lowered, remain the same, or be increased. Preferably, the anti-hyperlipidemic compound brings the serum levels of LDL cholesterol and HDL cholesterol (and, more preferably, triglyceride levels) to normal or nearly normal levels.

“Diuretic compound” is intended to mean any compound or agent that increases the amount of urine excreted by a patient.

“Neutral endopeptidase inhibitors” is intended to mean compounds that are antagonists of the renin angiotensin aldosterone system including compounds that are dual inhibitors of neutral endopeptidases and angiotensin converting (ACE) enzymes.

“Renin inhibitors” is intended to mean compounds which interfere with the activity of renin.

“Phosphodiesterase inhibitor” or “PDE inhibitor” is intended to mean any compound that inhibits the enzyme phosphodiesterase. The term refers to selective or non-selective inhibitors of cyclic guanosine 3′,5′-monophosphate phosphodiesterases (cGMP-PDE) and cyclic adenosine 3′,5′-monophosphate phosphodiesterases (cAMP-PDE).

“Platelet reducing agents” is intended to mean compounds that prevent the formation of a blood thrombus via any number of potential mechanisms. Platelet reducing agents include, but are not limited to, fibrinolytic agents, anti-coagulant agents and any inhibitors of platelet function. Inhibitors of platelet function include agents that impair the ability of mature platelets to perform their normal physiological roles, (i.e., their normal function, such as, for example, adhesion to cKIIunon-cellular entities, aggregation, release of factors such as growth factors) and the like.

“Proton pump inhibitor” refers to any compound that reversibly or irreversibly blocks gastric acid secretion by inhibiting the H.sup.+/K.sup.+-ATP ase enzyme system at the secretory surface of the gastric parietal cell.

“NSAID” is intended to mean a nonsteroidal anti-inflammatory compound or a nonsteroidal anti-inflammatory drug. NSAIDs inhibit cyclooxygenase, the enzyme responsible for the biosyntheses of the prostaglandins and certain autacoid inhibitors, including inhibitors of the various isozymes of cyclooxygenase (including but not limited to cyclooxygenase-1 and -2), and as inhibitors of both cyclooxygenase and lipoxygenase.

“Cyclooxygenase-2 (COX-2) selective inhibitor” is intended to mean a compound that selectively inhibits the cyclooxygenase-2 enzyme over the cyclooxygenase-1 enzyme. In one embodiment, the compound has a cyclooxygenase-2 IC.sub.50 of less than about 2 .mu.M and a cyclooxygenase-1 IC.sub.50 of greater than about 5 .mu.M, in the human whole blood COX-2 assay (as described in Brideau et al., Inflamm Res., 45: 68-74 (1996)) and also has a selectivity ratio of cyclooxygenase-2 inhibition over cyclooxygenase-1 inhibition of at least 10, and preferably 370 of at least 40. In another embodiment, the compound has a cyclooxygenase-1 IC.sub.50 of greater than about 1 .mu.M, and preferably of greater than 20 .mu.M. The compound can also inhibit the enzyme, lipoxygenase. Such selectivity may indicate an ability to reduce the incidence of common NSAT-induced side effects.

“Patient” is intended to mean animals, preferably mammals, most preferably humans, and includes males and females, and children and adults.

“Therapeutically effective amount” is intended to mean the amount of the compound and/or composition that is effective to achieve its intended purpose.

“Transdermal” is intended to mean the delivery of a compound by passage 385 through the skin and into the blood stream.

“Transmucosal” is intended to mean the delivery of a compound by passage of the compound through the mucosal tissue and into the blood stream.

“Penetration enhancement” or “permeation enhancement” is intended to mean an increase in the permeability of the skin or mucosal tissue to a selected pharmacologically active compound such that the rate at which the compound permeates through the skin or mucosal tissue is increased.

“Carriers” or “vehicles” are intended to mean carrier materials suitable for compound administration and include any such material known in the art such as, for example, any liquid, gel, solvent, liquid diluent, solubilizer, or the like, which is non-toxic and which does not interact with any components of the composition in a deleterious manner.

“Sustained release” is intended to mean the release of a therapeutically active compound and/or composition orally such that the blood levels of the therapeutically active compound are maintained within a desirable therapeutic range over a period of time. The sustained release formulation can be prepared using any conventional method known to one skilled in the art to obtain the desired release characteristics, such as a controlled release carrier comprised of a polymer selected from hydroxypropyl methylcellulose ethers or a combination of polymers selected from hydroxypropyl methylcellulose ethers and ethylcellulose, microencapsulated beads with specific permeation characteristics or degradation characteristics causing a predetermined release of active into the gastrointestinal tract.

Any device or step to a method described in this disclosure can comprise or consist of that which it is a part of, or the parts which make up the device or step. The term “and/or” is inclusive of the items which it joins linguistically and each item by itself. “Substantially” is defined as “at least 95% of the term being described” and any device or aspect of a device or method described herein can be read as “comprising” or “consisting” thereof.

### DETAILED DESCRIPTION OF EMBODIMENTS OF THE DISCLOSED TECHNOLOGY

Nitric oxide levels in the blood stream are augmented in a sustained manner while simultaneously delivering, in a normal or sustained release form, medications such as an NSAID or aspirin, hydroxyurea or renin/angiotension inhibitors as examples. This is accomplished by processing, at room temperature and/or in a low pressure environment without degradation, an amino acid ester with a nitric acid releasing group in a cellulose-based or other sustained release polymer. In this manner, in the blood stream nitric oxide levels are increased because the nitric oxide is released during hypertensive moments, either transient or prolonged, due to the metabolism of the nitric oxide releasing amino acid ester.

In embodiments of the disclosed technology, nitric oxide and it's metabolites are released in a sustained manner (defined as, “over a period of at least 3.5 hours”) and nitric oxide is created in response to hypertensive moments (defined as “periods of higher blood pressure of at least 10% above a resting level or blood pressure level over a period of not less than 2 minutes of resting”). Such hypertensive moments can be caused by eating, emotional changes, sitting up, or the like. The molecule, which will be described below, is activated based on induced hypertension, tightening of blood vessels, or higher flow of blood through a vessel. This concept of shear stress induced release of nitric oxide other than through the eNos system, was previously unknown to the inventor and appears to be unknown in the prior art, namely, that nitric oxide is released from these amino acid esters in hypertensive moments through upregulation of a endothelial glutathione transferase. By providing a sustained release of these nitric oxide releasing esters into the bloodstream a constant and increased supply of nitric oxide, nitrates and nitrites results that in the blood that remains, through a period of hours, with physiologically relevant effects throughout the body. Since endothelial function is a 24 hour a day physiological mechanism, releasing nitric oxide in response to stressors is optimal. A “stressor” is defined as “a chemical or biological agent, environmental condition, external stimulus or an event that causes increased pressure or tension within the circulatory system.” Dosing, in embodiments of the disclosed technology, is in the form of a 12 hour sustained release to be ingested or added to the blood stream twice daily with another medication (e.g. NSAID). In another embodiment, the dosing is once daily in a 24 hour sustained release form with the appropriate medications.

In some embodiments, single dosing of 15 to 30 milligrams (mg) of amino acid ester compounds with at least one nitric acid releasing group has been found to raise nitrates levels about 30 to 35% within five or seven days. This rise in circulating nitrates and nitrites is insufficient to completely reduce the gastro-toxic effects of ingested NSAIDs or have physiologically relevant effects in the treatment of medical conditions characterized by endothelial dysfunction or nitric oxide insufficiency. It was therefore necessary to find a way to consistently dose sufficient nitric oxide amino acid esters over an extended period of time in a minimum quantity necessary to release nitric oxide in a sustained manner and in such quantity as to achieve resultant nitrate and nitrite levels as set out previously herein-above. These levels have been shown to have positive physiologically effects, such as gastro-intestinal protection and cardiovascular health.

In order to achieve the metabolization of the nitric oxide releasing amino acid ester compounds, activation is triggered by the hypertensive state. When the blood vessels are restricted, endothelial glutathione transferase is up-regulated and the aforementioned amino acid ester compounds are metabolized in the endothelium. This metabolism is a two step process wherein the first step is a cleavage of the ester linkage by an esterase, subsequently the nitric oxide releasing moiety is metabolized by endothelial glutathione transferase. As blood vessels expand again, laminar flow decreases rapidly so that no matter what dosage is used, above the minimally required dosage for maximum release of nitric oxide, a similar response curve is realized for the metabolization of the molecule. By way of testing, the inventor has found the maximum dosage to be between about 0.15 mg/kg and 0.30 mg/kg each 1.5 to 2 hour period which is believed to be the half life of the nitric oxide releasing metabolic intermediate. Above this dosage amount and periodicity, in the prior art, no further substantial effect on nitric acid levels is found. It is further less than desirable to take medication every 1.5 to 2 hours to sustain the maximum effect, as required by the prior art.

While sustained release is known in the prior art, what is not previously known is how to accomplish sustained nitric oxide levels without risk or incidence of hypotension, development of tolerance and only as a response to hypertensive moments. In one example, aspirin is provided in about equal parts with an amino acid ester compound with at least one nitric acid releasing group. For purposes of this disclosure, “about” is defined as “within 15% thereof.” Each compound can be pre-created as a separate blend of materials to achieve the correct release characteristics, then blended together and tableted or put into capsules for dosing. For instance, aspirin is mixed with about two to three parts of hydroxyethylcellulose, about half a part of ethylcellulose, and about ⅕ a part of dibasic calcium phosphate dihydrate in one embodiment of the disclosed technology. In another example, in embodiments of the disclosed technology, 100 mg of a compound with a nitric oxide releasing amino acid ester is compounded into a similar mixture as above with reference to the aspirin, but with less hydroxyethylcellulose and more ethylcellulose. It is then mixed to create a sustained release mixture that can be combined in equal part of a drug to be delivered (e.g. aspirin). In such embodiments, 100 to 200 mg in a single compressed tablet or capsule can be delivered with a sustained release that lasts over a period of 4, 8, 12, or 24 hours.

The ethylcellulose (35 to 40 mg), in this embodiment, is dissolved with the aspirin or other drug in ethanol and mixed together. Then this mixture is dried and granulated. This can be done at low temperature (e.g. room temperature or below 30 degrees Celsius or below 25 degrees Celsius), or alternatively, produced at low pressure (less than 0.5, or 0.3 atmospheres) and results in about a 1:1 ratio of drug to delivered (e.g. aspirin) with the amino acid ester compound having a nitric oxide releasing amino acid ester In this manner, by way of use of alcohol and/or a vacuum/low pressure, the mixture is dried and granulated without degrading the nitric oxide releasing amino acid ester. It is within the scope of the invention that any sustained release formulation including those created with micro-encapsulation techniques such as those described in U.S. Pat. No. 5,603,957 can be utilized except that the use of hydro-alcoholic or alcoholic solvents for the nitric oxide releasing amino acid esters can not be used above 40 degrees Celsius, and in some embodiments, not above 35 degrees Celsius as the article quickly degrades under these conditions.

It should be understood that the use of alcohol at low temperature and pressure, in the present technology is another improvement over the prior art as no degradation of the nitric oxide releasing amino acid takes place due to the ability to prepare the mixture and dry at very low temperatures. Comparatively, in the prior art, using water and using temperatures above 40 degrees Celsius would result in quick degradation.

In a second example, naproxen or another NSAID or anti-inflammatory drug is used. In particular, naproxen is used for pain of osteoarthritis, but aside from it's gastric toxicity also increases blood pressure. Since naproxen has an approximate half life of 8 to 10 hours, this drug is taken normally twice a day for pain relief. Therefore, to advantageously combine this drug with a nitric oxide releasing amino acid ester, naproxen is, in embodiments of the disclosed technology, made into tablet form with a sustained release formulation of the nitric oxide releasing ester, as set out above, or in another instance, prepared as a capsule wherein the naproxen is mixed with the appropriate amount of sustained release microcapsules of the nitric oxide releasing amino acid ester to offset any gastrotoxicity or elevated blood pressure as a result of the naproxen dose. Nothing known in the prior art demonstrates the advantageous results from this sustained release of nitric oxide over intervals of time to allow unhindered delivery and release of the NSAID and blood pressure levels to be maintained at normal levels throughout the entire dosing regime while providing enhanced and virtually complete gastroenteral protection Similarly, many long acting drugs that could benefit from the sustained release of nitric oxide only in hypertensive moments so that hypotension is always avoided, could be contemplated and are within the scope and spirit of the disclosed technology. This is especially useful with in individuals with impaired endothelial function. Micro-encapsulated 100 mg nitric oxide releasing amino acid ester compound used, in embodiments of the disclosed technology, in a 12 hour sustained release format combined in a capsule formulation with 225 mg to 500 mg naproxen powder as a gastro protective blood pressure stable formulation is contemplated and part of the technology described herein. This method of dosing nitric oxide in a sustained release format is incapable of provoking hypotension and protects against the gastric toxicity of the NSAID partner while also offsetting any negative blood pressure effects of these agents.

Antioxidants such as Vitamin C or Vitamin E can be taken along with a nitric oxide releasing amino acid ester compound, at regular intervals, or daily in a sustained release form. In some embodiments, 0.15 to 0.30 mg of the amino acid ester compound per kilogram of patient is delivered via the sustained delivery form for the first 2 hours and from 0.05 to 0.15 mg/kg every hour thereafter for either 12 or 24 hours with sustained release aspirin. The sustained release is accomplished, in embodiments of the disclosed technology, by using a polymer selected from ethylcellulose, hydroxypropyl methylcellulose ethers, or other polymers such as known in the art or a combination of such polymers selected from hydroxypropyl methylcellulose ethers and ethylcellulose and others. The active ingredients are placed within or mixed with the sustained release compounds or formulated as micro-encapsulated beads similar to the processes as known in the art such as in U.S. Pat. No. 5,603,957 or using extruded lipid polymers such as glyceryl behenate.

Dosages of the nitric oxide releasing amino acid ester compound over 24 hours are, in embodiments of the disclosed technology, between 50 and 300 milligrams, such as specifically 150, 175, 225, or 250 milligrams, more preferably from 100 to 200 milligrams. When using aspirin, as described above, 160 milligrams of sustained release aspirin was combined with 175 milligrams of the nitric oxide releasing amino acid ester compound with a sustained release over 24 hours (releasing at least 95% or 99% of the active ingredient) and found to be effective in maintaining nitric oxide levels throughout the time period and over a period of 24 to 48 hours) when dosed twice, once a day, substantially raising and maintaining circulating levels of its metabolites nitrates and nitrites.

In further embodiments of the disclosed technology, an extruded (pushed through a die or cross-sectional shape) combination of a polymer and an amino acid ester with at least one nitric oxide releasing group are formed into a plurality of beads. The extrusion is carried out with, for example, castor wax (hydrogenated castor oil), another vegetable-based wax, or Compritol 888 (Glyceryl Behenate), a known pharmaceutical lipid used for extrusion. The amino acid ester releases the nitric oxide releasing group in a gastro-intestinal tract over a period between and including 4, 8, 12, 16, 20 and/or 24 hours. The release is planned/designed to happen as a result of shear stress in the intestinal tract which can be caused by hypertension or a hypertensive event. Such events cause nitric acid to be released which relaxes blood vessels and increases oxygen flow. As, in these embodiments of the disclosed technology, the nitric oxide releasing group is combined with an amino acid ester and a polymer physically mixed together (in a homogeneous or non-homogeneous manner).

Each episode of sheet stress and/or hypertension is configured to cause the release of some of the stored nitric acid, such as 5%, 10%, 12%, 15%, or 20% thereof due to the mechanical or chemical breakup of the nitric oxide releasing group, is subject to shear stress and/or hypertension during said period, said sheer stress and/or hypertension are self-regulated to release between 12% to 15% of stored nitric oxide per incidence of said shear stress and/or hypertension.

The extruded combination described above can be used to treat a normotensive or hypertensive patient with a nitric oxide deficiency or endothelial dysfunction. The combination, it should be understood, can be extruded or formed in any other manner and is considered to be the equivalent thereof so long as the resultant combination is created and dosed for being taken internally into the body. Liberation of nitric oxide from the nitric oxide releasing group through a glutathione transferase dependent reaction relieves hypertension in embodiments of the disclosed technology.

In other embodiments, a medication being administered to a patient causing an increase in nitric oxide levels in the blood of the patient and/or toxicity. “Medication” for purposes of this disclosure is “any substance used to treat or change conditions in a living organism.” The medication, in embodiments of the disclosed technology, has a limited duration use per period of time before becoming harmful, dangerous, or over recommended usage levels by a government agency or manufacturer thereof. The addition of the compound described above, the sustained release amino acid and nitric oxide releasing ester, improves efficacy (desired result) of the medication and usability over thereof over the limited duration use per period of time, and in embodiments of the disclosed technology, over a second longer duration which is longer than said first shorter and limited duration of time. Thus, the sustained release of nitric oxide, in such embodiments of the disclosed technology, is used to allow another and separate medication given in a same or different actual dose, extrusion, or tablet, to be more effective and used in greater doses with the disclosed technology than previously able to do so, or able to be administered by itself.

To be more specific, in some embodiments, the compound is administered in conjunction with the other/additional medication and dosed, based on expected hypertensive episodes, to remain in a body of someone taking the medication for a period of at least 4 hours and in some cases, as many as 24 hours or any time frame there-between as described above.

The above or another medication which is administered has an anti-thrombotic effect unrelated to cGMP (cyclic guanosine 3′,5′-monophosphate phosphodiesterases), wherein the addition of the sustained release amino acid nitric oxide releasing ester has a synergistic anti-thrombotic effect with the medication.

The above-described increases in hypertension or sheer stress can be related to normal daily activities. “Normal daily activities” are defined as those carried out by what a physician considers a healthy patient carrying out activities throughout the day which the patient carries out at least 10 times a year. In some embodiments, “normal daily activities” include at least some of walking, working at a job, putting on clothing, house work, phone calls, talking, watching TV, resting, and sleeping.

The beads can be coated with an enteric coating such as ethyl cellulose and triacetin (triglyceride 1,2,3-triacetoxypropane) or a coating with methacrylate copolymers or derivatives thereof so that the component parts are digested in the intestine. The coating can be Eudragit L100 55 (an anionic copolymer based on methacrylic acid and ethyl acrylate). The methacrylate copolymer coating dissolves above pH 5.5 at the entry to the small intestine.

The nitric acid releasing compound can be used by determining/adjusting a ratio of polymer to amino acid ester based on what was determined in prior clinical, observed, or interpolated (from prior results) data such that the nitric oxide is released over a length of time substantially equal (within 25% plus or minus) or equal to that of a half life of another medication administered with the compound. The compound is then administered/dispensed/dosed to a patient with the additional other medication. Hypertensive events caused by the another medication are then mitigated (counteracted) by sustained release of the nitric oxide over at least a majority of time equal to that of the half life of the another medication.

The extrusion described above can be conducted under substantially anhydrous conditions which can prevent hydrolyzation of the ester during the extrusion. This can be done at a temperature of 70 degrees or below.

The medication described above can be one that treats hypercholesterolemia, diabetes, hypertension, red blood cell cycling, platelet adhesion, stroke, any other diseases or states effected by endothelial dysfunction or nitric oxide insufficiencies.

For example, hydroxyurea is a nitric oxide donor used to treat sickle cell and beta thalassrmia through increasing fetal hemoglobin via nitric oxide. Hydroxyurea, however, is toxic and can only be given once a day and has only about a two hour half life. Using the sustained release compound in conjunction with a drug like hydroxyurea a 24 hour release profile of nitric oxide is attained thus improving fetal hemoglobin production, decreasing platelet adhesion over a full 24 hours and improving clinical outcome. f

f

In another example, Pre-diabetics are normotensive but have endothelial dysfunction. Combining compounds of the disclosed technology which release nitric oxide during hypertensive events over a 24 hour period of time along with a medication such as low dose aspirin or aspirin which lasts over a period of 24 hours (in one or multiple doses) decreases the risk of stroke. Nitric oxide is released due to the periodic hypertensive bursts through normal activity.

In conclusion, it has been found that a single dose of a nitric oxide releasing amino acid ester compound is ineffective to significantly induce nitric oxide release in individuals to achieve therapeutic levels since dose response is not correlated to blood pressure increase nor to dosage above a maximum amount e.g. approximately 25 mg in a 200 lb individual.

Therefore, only a sustained release dosage form of the nitric oxide releasing amino acid ester can effect significant nitric oxide release in both normo-tensive and hypertensive individuals. It had been advantageously discovered that even the hypertensive response to feeding produces a rapid upregulation in the metabolism of the nitric oxide releasing compounds quickly offsetting any increase in blood pressure and liberating a small (10% as measured by increase of nitrate/nitrites) amount of nitric oxide. Thus throughout the normal course of a day for an ordinary individual there will likely be at least three hypertensive events in a non-amublatory individual excluding emotional stressor events. In a normal ambulatory individual, there can be normally five or six transient hypertensive events daily with the concurrent release of nitric oxide in embodiments of the disclosed technology. In all these cases of sustained release, physiologically relevant levels of nitric oxide will be released over 24 to 48 hours with the formation of physiologically relevant levels of its metabolites such as nitrates and nitrites.

In hypertensive populations, such as those with type 2 diabetes, a more sustained release of nitric oxide should be attained in the first twelve hours with consequent formation of the relevant and beneficial metabolites such as nitrate and nitrite.

While the disclosed technology has been taught with specific reference to the above embodiments, a person having ordinary skill in the art will recognize that changes can be made in form and detail without departing from the spirit and the scope of the disclosed technology. The described embodiments are to be considered in all respects only as illustrative and not restrictive. All changes that come within the meaning and range of equivalency of the claims are to be embraced within their scope. Combinations of any of the methods, systems, and devices described herein-above are also contemplated and within the scope of the disclosed technology.

1. A compound delivery system, comprising:
an extruded combination comprising a polymer and an amino acid ester with at least one nitric oxide releasing group formed into a plurality of beads, said extruded combination is formed at a temperature equal to or below 70 degrees Celsius,
wherein said amino acid ester releases said nitric oxide releasing group in a gastro intestinal tract over a period between and including 4 to 24 hours, such that by the end of said period at least 95% of said nitric oxide releasing group in said extruded combination has been released; and
wherein said nitric oxide releasing group is selected such that once said extruded combination is absorbed into the blood stream, when said nitric oxide releasing group is subject to shear stress and/or hypertension during said period, said sheer stress and/or hypertension self-regulate to release of between 12% to 15% of stored nitric oxide per incidence of said shear stress and/or hypertension.

2. A method of using the compound of claim 1, comprising administering said compound to treat a normotensive or hypertensive patient with a nitric oxide deficiency or endothelial dysfunction.

3. A method of using the compound of claim 1, comprising:
administering a medication which causes hypertension;
using said compound to prevent said hypertension and/or tolerance of said medication due to liberation of said nitric oxide from said nitric oxide releasing group through a glutathione transferase dependent reaction.

4. A method of using the compound of claim 1, comprising administering a medication causing at least one of a increase in nitric oxide levels and/or toxicity, said medication usable by itself for a first shorter duration, wherein addition of said sustained release amino acid nitric oxide releasing ester improves efficacy of said medication and usability over a second longer duration which is longer than said first shorter duration.

5. A method of using the compound of claim 1, comprising administering a medication with an anti-thrombotic effect unrelated to cGMP, wherein the addition of said sustained release amino acid nitric oxide releasing ester has a synergistic anti-thrombotic effect with said medication.

6. A method of using the compound of claim 1, in a sustained release form over a time period of at least 4 hours, wherein said release from the nitric oxide releasing group is in response to increases in blood pressure or shear stress related to normal daily activities.

7. (canceled)

8. The compound delivery system of claim 19, wherein said fat containing molecule is glyceryl behenate.

9. (canceled)

10. The compound delivery system of claim 1, wherein said period is 24 hours.

11. The compound delivery system of claim 1, wherein said plurality of beads are coated with an enteric coating, comprising methacrylate copolymers or derivatives thereof.

12. The compound delivery system of claim 11, wherein said enteric coating is poly(methacrylic acid, ethyl acrylate) 1:1, wherein said methacrylate copolymer coating dissolves above pH 5.5 at the entry to the small intestine.

13. The compound delivery system of claim 11, wherein said nitric oxide releasing amino acid ester is released in the small intestine due in part to presence of said enteric coating.

14. A method of using the compound of claim 1, comprising the steps of:
adjusting a ratio of polymer to amino acid ester, based on prior determinations of ratios, such that said nitric oxide is released over a length of time substantially equal to that of a half life of another medication administered with said compound;
administering said compound and said another medication to a patient;
wherein hypertensive events caused by said another medication are counteracted by sustained release of said nitric oxide over at least a majority of time equal to that of said half life of said another medication.

15. The method of using the compound claim 11, wherein said at least a majority of time is substantially a time of said half life of said another medication.

16. The compound delivery system of claim 1, wherein extrusion of said extruded combination is conducted under substantially anhydrous conditions.

17. The compound delivery system of claim 11, wherein said amino acid ester lacks hydrolyzation during said extrusion.

18. (canceled)

19. A compound delivery system, comprising:
an extruded combination comprising a fat containing molecule and an amino acid ester with at least one nitric oxide releasing group formed into a plurality of beads;
wherein said amino acid ester releases said nitric oxide releasing group in a gastro intestinal tract over a period between and including 4 to 24 hours, such that by the end of said period at least 95% of said nitric oxide releasing group in said extruded combination has been released; and
wherein said nitric oxide releasing group is selected such that once said extruded combination is absorbed into the blood stream, when said nitric oxide releasing group is subject to shear stress and/or hypertension during said period, said sheer stress and/or hypertension self-regulate release of between 12% to 15% of stored nitric oxide per incidence of said shear stress and/or hypertension.
